(19) United States
(12) Patent Application Publication
(10) Pub. No.: US 2023/0241395 A1
(43) Pub. Date: Aug. 3, 2023

(54) CONTROL OF MEDICAL DEVICE USING REPRESENTATIVE VALUE

(71) Applicant: Medtronic, Inc., Minneapolis, MN (US)

(72) Inventors: Todd D. Zenisek, Georgetown, TX (US); Juan G. Hincapie, Maple Grove, MN (US); Andrew J. Cleland, St. Paul, MN (US); Leonid M. Litvak, Beit Shemesh (IL); Andrew L. Schmeling, Holmen, WI (US)

(21) Appl. No.: 18/066,083

(22) Filed: Dec. 14, 2022

Related U.S. Application Data
(60) Provisional application No. 63/267,433, filed on Feb. 2, 2022.

(51) Int. Cl. A61N 1/36; A61N 1/372

(52) U.S. Cl. CPC A61N 1/36132; A61N 1/37223; A61N 1/36139

(57) ABSTRACT

A system includes telemetry circuitry configured for communication between a medical device and an external device associated with the medical device and processing circuitry. The processing circuitry is configured to receive an indication of a plurality of user inputs, each user input of the plurality of user inputs indicating a respective value of a plurality of values for a stimulation parameter that at least partially defines therapy provided to the patient in a posture state of a plurality of posture states. The processing circuitry is further configured to determine a representative value for the stimulation parameter based on the plurality of values for the stimulation parameter that at least partially defines therapy provided to the patient in the posture state. The processing circuitry is further configured to control the medical device to provide the therapy according to the representative value.

This application claims the benefit of U.S. Provisional Patent Application No. 63/267,433, filed 2 Feb. 2022, the entire contents of which is incorporated herein by reference.

### TECHNICAL FIELD

This disclosure generally relates to medical devices, and, more specifically, medical device configured to deliver therapy to a patient.

### BACKGROUND

Medical devices (e.g., an implantable medical device or an external medical device) may include electrical stimulation devices, drug pumps, insulin pumps, or cardiac stimulation devices. Electrical stimulation devices, for example, neurostimulators or neurostimulation devices, may be external to or implanted within a patient, and configured to deliver electrical stimulation therapy to various tissue sites to treat a variety of symptoms or conditions such as chronic pain, tremor, Parkinson's disease, epilepsy, or other neurological disorders, urinary or fecal incontinence, sexual dysfunction, obesity, or gastroparesis. An electrical stimulation device may deliver electrical stimulation therapy via electrodes, e.g., carried by one or more leads, positioned proximate to target locations associated with the brain, the spinal cord, pelvic nerves, tibial nerves, peripheral nerves, the gastrointestinal tract, or elsewhere within a patient. Stimulation proximate the spinal cord, proximate the sacral nerve, within the brain, and proximate peripheral nerves is often referred to as spinal cord stimulation (SCS), sacral neuromodulation (SNM), deep brain stimulation (DBS), and peripheral nerve stimulation (PNS), respectively.

### SUMMARY

In general, this disclosure describes techniques for training a medical device to provide therapy to a patient. The medical device may be implantable and/or wearable and may be configured to provide one or more of deep brain stimulation (DBS), spinal cord stimulation (SCS), sacral neuromodulation (SNM), and peripheral nerve stimulation (PNS), targeted drug delivery (TDD), or another therapy. For example, a system may be configured to determine a representative value for a stimulation parameter based on a plurality of values for that stimulation parameter that were used to at least partially define therapy provided to a patient in a posture state. In this example, the medical device may then deliver subsequent therapy using the representative value for the stimulation parameter when the patient is in the posture state.

In one example, this disclosure is directed to a system comprising telemetry circuitry configured for communication between a medical device and an external device associated with the medical device and processing circuitry. The processing circuitry is configured to receive an indication of a plurality of user inputs, each user input of the plurality of user inputs indicating a respective value of a plurality of values for a stimulation parameter that at least partially defines therapy provided to the patient in a posture state of a plurality of posture states. The processing circuitry is further configured to determine a representative value for the stimulation parameter based on the plurality of values for the stimulation parameter that at least partially defines therapy provided to the patient in the posture state. The processing circuitry is further configured to control the medical device to provide the therapy according to the representative value for the stimulation parameter when the patient is in the posture state.

In another example, this disclosure is directed to a method comprising receiving, by one or more processors, an indication of a plurality of user inputs, each user input of the plurality of user inputs indicating a respective value of a plurality of values for a stimulation parameter that at least partially defines therapy provided to the patient in a posture state of a plurality of posture states. The method further includes determining, by the one or more processors, a representative value for the stimulation parameter based on the plurality of values for the stimulation parameter that at least partially defines therapy provided to the patient in the posture state. The method further includes controlling, by the one or more processors, the medical device to provide the therapy according to the representative value for the stimulation parameter when the patient is in the posture state.

In one example, this disclosure is directed to a computer-readable storage medium having stored thereon instructions that, when executed, cause processing circuitry to receive an indication of a plurality of user inputs, each user input of the plurality of user inputs indicating a respective value of a plurality of values for a stimulation parameter that at least partially defines therapy provided to the patient in a posture state of a plurality of posture states. The instructions further cause processing circuitry to determine a representative value for the stimulation parameter based on the plurality of values for the stimulation parameter that at least partially defines therapy provided to the patient in the posture state. The instructions further cause processing circuitry to control the medical device to provide the therapy according to the representative value for the stimulation parameter when the patient is in the posture state.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF DRAWINGS

FIG. 1 is a conceptual diagram illustrating an example system that includes an implantable medical device (IMD) in the form of a neurostimulation device configured to deliver spinal cord stimulation (SCS) and an external programmer, in accordance with one or more techniques of this disclosure.

FIG. 2 is a block diagram illustrating an example of an IMD in the form of a stimulation device, in accordance with one or more techniques of this disclosure.

FIG. 3 is a block diagram illustrating an example of an external programmer suitable for use with the IMD of FIG. 2, in accordance with one or more techniques of this disclosure.

FIG. 4 is a flow diagram illustrating a process for determining a representative value for a stimulation parameter that at least partially defines a therapy provided to a patient, in accordance with one or more techniques of this disclosure.

### DETAILED DESCRIPTION

This disclosure describes techniques, devices, and systems for training a medical device to deliver therapy to a patient. In one example, an external device (e.g., an external programmer and/or a medical device) may be configured to train the medical device to provide therapy to a patient based on various obtained data such as previously used parameter values that define the therapy. Currently, patients may have to come to a medical office and have their healthcare provider program and titrate (e.g., set or change) each stimulation parameter that at least partially defines therapy provided to the patient. Healthcare providers, or representatives (reps), have to spend a significant amount of time to perform programming optimization for each medical device. For pain therapy, a system may adjust the stimulation parameters to certain parameter values for a given specific position state. For example, if the patient desires to adjust the stimulation parameter in a given position state to improve therapy efficacy in that posture state, the patient may either adjust the stimulation parameter themselves or a healthcare professional or representative may make an adjustment to a specific stimulation parameter or set of stimulation parameters. These manual adjustments may be a burden for the patient and/or healthcare professional in term of time, effort, and inadvertent adjustment errors. In addition, each adjustment may require interaction with an external programmer. Moreover, each time the patient interacts with their programmer and/or medical device, the interaction may remind the patient that they have an implant and a disease, which may reduce a satisfaction with the therapy provided by the medical device.

In accordance with the techniques of the disclosure, a system may provide a “learning” algorithm that will automatically determine a representative value for one or more parameters that at least partially define subsequent stimulation. For example, the system may average “X” number of titrations (e.g., patient requested changes) in a specific posture state and use that average value for the parameter in subsequent stimulation. This average value may provide a better, patient specific, programming experience compared to systems that rely on the patient and/or healthcare provider to determine an appropriate value for the parameter. In one example, the system may average all titrations for a specific posture state (e.g., identified by a 3-axis accelerometer in the medical device), which may greatly reduce frequency that the patient needs to adjust the stimulation parameter value each time when re-entering the posture state.

Due to this learning algorithm, the system may omit a manual calibration of the system of what the different body posture states are, which may save programming time. For example, the system may allow the patient to select a set of stimulation parameter values that includes one or more of an amplitude, a pulse width, or rate, and the system may generate a representative value for each stimulation parameter based on these selected values over time. For instance, the system may auto average the titrations for each of one or more body posture state. For example, when the patient is laying down, the system may receive user input over time indicating a titration of the amplitude 10 times having respective different values (e.g., 2.0 milliamps (mA), 2.2 mA, 2.1 mA, 2.4 mA, 2.6 mA, etc.). In this example, the system may average those amplitude values to generate a representative value. The next time the patient is in the given body posture state, the system may provide the representative amplitude value of 2.3 mA. In some examples, the system may use a standard number of values used for averaging (e.g. rolling average of the last 10 titrations). In other examples, the system may use a weighted average, median value, average with dropped outlier values, or any other representative value prior parameter values. Additionally, or alternatively, the system may enable the healthcare provider to provide input to set the number of values used for averaging and/or the averaging method used. In this way, the system may help to provide patient control measures that simplify or make a setup or changes to a therapy easier for a patient, which may improve a therapy provided to the patient. Moreover, the patient control measures may reduce a number of times that a clinician need to intervene to select parameter values for the system, which may help to reduce an amount of time a clinician spends configuring medical devices. In this way, the system may help to provide patient control measures that simplify or make changes to a therapy easier for a patient, in addition to providing more appropriate parameter values for therapy, which may improve a therapy provided to the patient.

Techniques described herein may be directed to implantable medical devices and external medical devices. Examples disclosed herein may describe techniques with reference to specific medical devices such as implantable neurostimulators; however, aspects of such techniques may apply to any medical device. Again, examples of medical devices, which may be external or implantable, may include drug pumps, insulin pumps, or cardiac stimulation devices.

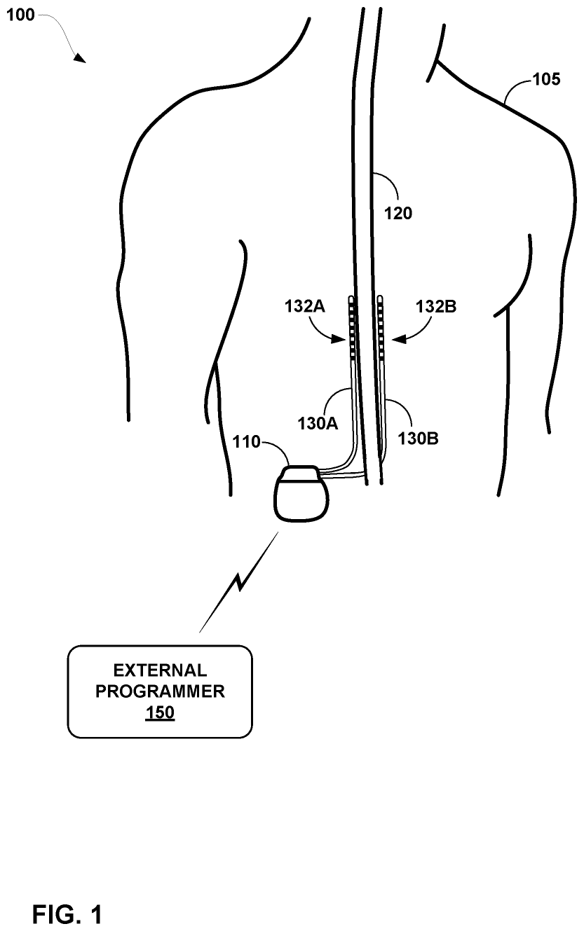

FIG. 1 is a conceptual diagram illustrating an example system 100 that includes an implantable medical device (IMD) 110 configured to deliver SCS therapy, processing circuitry 140, and an external programmer 150, in accordance with one or more examples of this disclosure. Although the examples described in this disclosure are generally applicable to a variety of medical devices including external devices and IMDs, application of such techniques to IMDs and, more particularly, implantable electrical stimulators (e.g., neurostimulators) will be described for purposes of illustration. More particularly, the disclosure will refer to an implantable SCS system for purposes of illustration, but without limitation as to other types of medical devices or other therapeutic applications of stimulation.

External programmer 150 may be configured to provide a “learning” algorithm that will automatically determine a representative value for one or more parameters that at least partially define subsequent stimulation. For example, external programmer 150 may average “X” number of titrations (e.g., patient requested changes) in a specific posture state and use that average value for the parameter in subsequent stimulation. This average value may provide a better, patient specific, programming experience compared to systems that rely on the patient and/or healthcare provider to determine an appropriate value for the parameter.

As shown in FIG. 1, system 100 includes an IMD 110, leads 130A and 130B, and external programmer 150 shown in conjunction with a patient 105, who is ordinarily a human patient. In the example of FIG. 1, IMD 110 is an implantable electrical stimulator that is configured to generate and deliver electrical stimulation therapy to patient 105, e.g., for relief of chronic pain or other symptoms, or restoration or support of physical function or control in the case of spinal cord injury or degeneration, via one or more electrodes 132A, 132B of leads 130A and/or 130B, respectively. In the example of FIG. 1, each lead 130A, 130B includes eight electrodes 132A, 132B respectively, although the leads may each have a different number of electrodes. Leads 130A, 130B may be referred to collectively as “leads 130” and electrodes 132A, 132B may be referred to collectively as electrodes 132. In other examples, IMD 110 may be coupled to a single lead carrying multiple electrodes or more than two leads each carrying multiple electrodes.

IMD 110 may be a chronic electrical stimulator that remains implanted within patient 105 for weeks, months, or years. In other examples, IMD 110 may be a temporary, or trial, stimulator used to screen or evaluate the efficacy of electrical stimulation for chronic therapy. In one example, IMD 110 is implanted within patient 105, while in another example, IMD 110 is an external device coupled to one or more leads percutaneously implanted within the patient. In some examples, IMD 110 uses electrodes on one or more leads, while in other examples, IMD 110 use one or more electrodes on a lead or leads and one of more electrodes on a housing of the IMD. In further examples, IMD 110 may be leadless and instead use only electrodes carried on a housing of IMD.

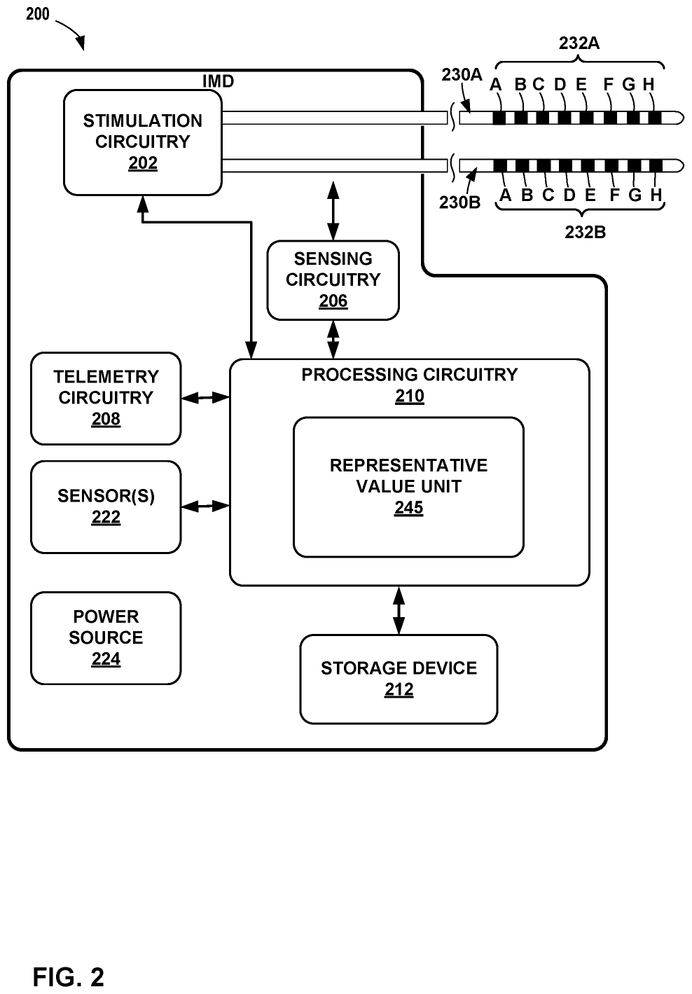

IMD 110 may be constructed of any polymer, metal, or composite material sufficient to house the components of IMD 110 (e.g., components illustrated in FIG. 2) within patient 105. In this example, IMD 110 may be constructed with a biocompatible housing, such as titanium or stainless steel, or a polymeric material such as silicone, polyurethane, or a liquid crystal polymer, and surgically implanted at a site in patient 105 near the pelvis, abdomen, or buttocks. In other examples, IMD 110 may be implanted at other suitable sites within patient 105, which may depend, for example, on the target site within patient 105 for the delivery of electrical stimulation therapy. The outer housing of IMD 110 may be configured to provide a hermetic seal for components, such as a rechargeable or non-rechargeable power source. In addition, in some examples, the outer housing of IMD 110 is selected from a material that facilitates receiving energy to charge the rechargeable power source.

In the example of FIG. 1, electrical stimulation energy, which may be delivered as regulated current or regulated voltage-based pulses, is delivered from IMD 110 to one or more target tissue sites of patient 105 via leads 130 and electrodes 132. Leads 130 position electrodes 132 adjacent to target tissue of spinal cord 120. One or more of the electrodes 132 may be disposed at a distal tip of a lead 130 and/or at other positions at intermediate points along the lead. Leads 130 may be implanted and coupled to IMD 110. The electrodes 132 may transfer electrical stimulation generated by an electrical stimulation generator in IMD 110 to tissue of patient 105. Although leads 130 may each be a single lead, a lead 130 may include a lead extension or other segments that may aid in implantation or positioning of lead 130.

The electrodes of leads 130 may be electrode pads on a paddle lead, circular (e.g., ring) electrodes surrounding the body of the lead, conformable electrodes, cuff electrodes, segmented electrodes (e.g., electrodes disposed at different circumferential positions around the lead instead of a continuous ring electrode), any combination thereof (e.g., ring electrodes and segmented electrodes) or any other type of electrodes capable of forming unipolar, bipolar or multipolar electrode combinations for therapy. Ring electrodes arranged at different axial positions at the distal ends of lead 130 will be described for purposes of illustration. Deployment of electrodes via leads 130 is described for purposes of illustration, but electrodes may be arranged on a housing of IMD 110, e.g., in rows and/or columns (or other arrays or patterns), as surface electrodes, ring electrodes, or protrusions.

Stimulation parameters defining the electrical stimulation pulses delivered by IMD 110 through electrodes 132 of leads 130 may include information identifying which electrodes have been selected for delivery of the stimulation pulses according to a stimulation program and the polarities of the selected electrodes (the electrode combination), and voltage or current amplitude, pulse rate (e.g., frequency), and pulse width of the stimulation pulses. The stimulation parameters may further include a cycle parameter that specifies when, or how long, stimulation is turned on and off. Stimulation parameters may be programmed prior to delivery of the stimulation pulses, manually adjusted based on user input, or automatically controlled during delivery of the stimulation pulses, e.g., based on sensed conditions.

Although the example of FIG. 1 is directed to SCS therapy, e.g., to treat pain or restore or support physical function or control in the case of spinal cord injury or degeneration, in other examples, system 100 may be configured to treat other conditions that may benefit from stimulation therapy. For example, system 100 may be used to treat tremor, Parkinson's disease, epilepsy, or other neurological disorders, urinary or fecal incontinence, sexual dysfunction, obesity, or gastroparesis, or psychiatric disorders such as depression, mania, obsessive compulsive disorder, or anxiety disorders. Hence, in some examples, system 100 may be configured to deliver SNM, DBS, PNS, or other stimulation, such as peripheral nerve field stimulation (PNFS), cortical stimulation (CS), gastrointestinal stimulation, or any other stimulation therapy capable of treating a condition of patient 105.

Leads 130 may include, in some examples, one or more sensors configured to sense one or more physiological parameters of patient 105, such as patient activity, pressure, temperature, or other characteristics. At least some of electrodes 132 may be used to sense electrical signals within patient 105, additionally or alternatively to delivering stimulation. IMD 110 is configured to deliver electrical stimulation therapy to patient 105 via selected combinations of electrodes carried by one or both of leads 130, alone or in combination with an electrode carried by or defined by an outer housing of IMD 110. The target tissue for the electrical stimulation therapy may be any tissue affected by electrical stimulation. In some examples, the target tissue includes nerves, smooth muscle or skeletal muscle. In the example illustrated by FIG. 1, the target tissue is tissue proximate spinal cord 120, such as within an intrathecal space or epidural space of spinal cord 120, or, in some examples, adjacent nerves that branch off spinal cord 120. Leads 130 may be introduced into spinal cord 120 in via any suitable region, such as the thoracic, cervical or lumbar regions.

Stimulation of spinal cord 120 may, for example, prevent pain signals from traveling through spinal cord 120 and to the brain of patient 105. Patient 105 may perceive the interruption of pain signals as a reduction in pain and, therefore, efficacious therapy results. In other examples, stimulation of spinal cord 120 may produce paresthesia which may reduce the perception of pain by patient 105, and thus, provide efficacious therapy results. In some examples, some electrical stimulation pulses may be directed to glial cells while other electrical stimulation (e.g., delivered by a different electrode combination) is directed to neurons. In other examples, electrical stimulation pulses may be directed to restore a function lost due to a spinal cord injury.

IMD 110 may generate and may deliver electrical stimulation therapy to a target stimulation site within patient 105 via the electrodes of leads 130 to patient 105 according to one or more therapy stimulation programs. A therapy stimulation program specifies values for one or more parameters that define an aspect of the therapy delivered by IMD 110 according to that program. For example, a therapy stimulation program that controls delivery of stimulation by IMD 110 in the form of stimulation pulses may define values for voltage or current pulse amplitude, pulse width, and pulse rate (e.g., pulse frequency) for stimulation pulses delivered by IMD 110 according to that program, as well as the particular electrodes and polarities forming an electrode combination used to deliver the stimulation pulses.

A user, such as a clinician, caretaker, or patient 105, may interact with a user interface of an external programmer 150 to program IMD 110. External programmer 150 may represent a physician programmer or patient programmer. Programming of IMD 110 may refer generally to the generation and transfer of commands, programs, or other information to control the operation of IMD 110. In this manner, IMD 110 may receive the transferred commands and programs from external programmer 150 to control electrical stimulation therapy. External programmer 150 may transmit therapy stimulation programs, program groups, stimulation parameter adjustments, therapy stimulation program selections, user input, or other information to control the operation of IMD 110, e.g., by wireless telemetry or wired connection.

External programmer 150 may perform a stimulation parameter adjustment that changes a set of stimulation parameters of an existing program. For example, external programmer 150 may automatically, semi-automatically, or based on a user selection, may determine or more stimulation parameter adjustments for an existing program. In this example, external programmer 150 may pass through the one or more parameter adjustments for the existing program. For instance, external programmer 150 may determine a parameter adjustment (e.g., receive the adjustment from a user input from a health professional) that sets an intensity value of a particular stimulation parameter of a program and may relay the parameter adjustment to IMD 110.

External programmer 150 may be characterized as a physician or clinician programmer if external programmer 150 is primarily intended for use by a physician or clinician. In other cases, external programmer 150 may be characterized as a patient programmer if external programmer 150 is primarily intended for use by a patient. A patient programmer may be generally accessible to patient 105 and, in many cases, may be a portable device that may accompany patient 105 throughout the patient's daily routine. For example, a patient programmer may receive input from patient 105 when the patient wishes to terminate or change stimulation therapy. In general, a physician or clinician programmer may support selection and generation of programs by a clinician for use by IMD 110, whereas a patient programmer may support adjustment and selection of such programs by a patient during ordinary use. In other examples, external programmer 150 may include, or be part of, an external charging device that recharges a power source of IMD 110. In this manner, a user may program and charge IMD 110 using one device, or multiple devices.

IMD 110 and external programmer 150 may exchange information and may communicate via wireless communication. Examples of communication techniques may include, for example, radiofrequency (RF) telemetry and inductive coupling, but other techniques are also contemplated. In some examples, external programmer 150 includes a communication head that may be placed proximate to the patient's body near the IMD 110 implant site to improve the quality or security of communication between IMD 110 and external programmer 150. Communication between external programmer 150 and IMD 110 may occur during power transmission or separate from power transmission.

IMD 110, in response to commands from external programmer 150, may deliver electrical stimulation therapy according to one or more therapy stimulation programs, or a group of programs to a target tissue site of the spinal cord 120 of patient 105 via electrodes 132 on leads 130. In some examples, IMD 110 automatically modifies therapy stimulation programs as therapy needs of patient 105 evolve over time. For example, the modification of the therapy stimulation groups or programs may cause the adjustment of at least one parameter of the plurality of stimulation pulses.

In accordance with the techniques of the disclosure, external programmer 150 may receive an indication of a plurality of user inputs, each user input of the plurality of user inputs indicating a respective value of a plurality of values for a stimulation parameter that at least partially defines therapy provided to the patient in a posture state of a plurality of posture states. For example, patient 105 may interact with a touchscreen of external programmer 150 over time to specify a titration (or changes) of the amplitude N number of times (e.g., where N is a positive integer) while the patient is in a laying down posture state of a plurality of different posture states (e.g., sitting, standing, walking, or laying down in different positions such as supine and prone posture states). Each amplitude adjustment may occur during a single continuous duration in the same posture state or over different instances of the posture state broken up by the patient assuming different posture states in between instances of the posture state. The patient may provide fewer or greater changes to the amplitude. External programmer 150 may determine a representative value for the stimulation parameter based on the plurality of values for the stimulation parameter that at least partially defines therapy provided to patient 105 in the posture state of the plurality of posture states. For example, external programmer 150 may average the titrations of the amplitude a number of times to an amplitude of 2.3 mA. External programmer 150 may control IMD 110 to provide the therapy according to the representative value for the stimulation parameter when patient 105 is in the posture state. For example, external programmer 150 may control, based on a determination that patient 105 is laying down, IMD 110 to provide therapy with an amplitude of 2.3 mA. While this example refers to external programmer 150 determining a representative value, in other examples other components of system 100 and/or other components may determine the representative value. For example, IMD 110 may determine the representative value and control itself to provide therapy using the representative value. These techniques may apply to two or more posture states of the plurality of posture states, such that IMD 110 may apply respective representative values for multiple or all of the posture states.

FIG. 2 is a block diagram illustrating an example configuration of components of an IMD 200, in accordance with one or more techniques of this disclosure. IMD 200 may be an example of IMD 110 of FIG. 1. In the example shown in FIG. 2, IMD 200 includes stimulation circuitry 202, sensing circuitry 206, telemetry circuitry 208, processing circuitry 210, storage device 212, sensor(s) 222, power source 224, lead 230A carrying electrodes 232A, which may correspond to lead 130B and electrodes 132B of FIG. 1, and lead 230B carrying electrodes 232B, which may correspond to lead 130B and electrodes 132B of FIG. 1.

Stimulation circuitry 202 may generate electrical stimulation pulses selected to alleviate symptoms or dysfunction of one or more diseases, disorders, injuries, or syndromes. Representative value unit 245 may be configured to determine a representative value for a stimulation parameter (e.g., an amplitude, pulse width, frequency, or any other parameter that may contribute to the intensity of the electrical stimulation pulses). Intensity may comprise a function of amplitude, pulse width, and/or frequency of the electrical stimulation pulses. The patient may directly adjust one of these parameters and/or the intensity as a whole in some examples. While stimulation pulses are described, stimulation signals may take other forms, such as continuous-time signals (e.g., sine waves) or the like. Each of leads 230A, 230B may include any number of electrodes 232A, 232B. In the example of FIG. 2, each set of electrodes 232A, 232B includes eight electrodes A-H. In some examples, the electrodes are arranged in bipolar combinations. A bipolar electrode combination may use electrodes carried by the same lead 230A, 230B or different leads. For example, an electrode A of electrodes 232A may be a cathode and an electrode B of electrodes 232A may be an anode, forming a bipolar combination.

Each of the electrodes 232A, 232B may be associated with respective regulated current source and sink circuitry to selectively and independently configure the electrode to be a regulated cathode or anode. In this way, current sourced or sunk by selected electrodes may be individually controlled. In some examples, IMD 200 may include switch circuitry that include one or more switch arrays, one or more multiplexers, one or more switches (e.g., a switch matrix or other collection of switches), or other electrical circuitry configured to direct stimulation signals from stimulation circuitry 202 to one or more of electrodes 232A, 232B, or directed sensed signals from one or more of electrodes 232A, 232B to sensing circuitry 206. Stimulation circuitry 202 and/or sensing circuitry 206 also may include sensing circuitry to direct electrical signals sensed at one or more of electrodes 232A, 232B.

Sensing circuitry 206 may be configured to monitor signals from any combination of electrodes 232A, 232B. In some examples, sensing circuitry 206 includes one or more amplifiers, filters, and analog-to-digital converters. Sensing circuitry 206 may be used to sense electrophysiological signals. In some examples, sensing circuitry 206 detects electrophysiological signals from a particular combination of electrodes 232A, 232B. In some cases, the particular combination of electrodes for sensing electrophysiological signals includes different electrodes than a set of electrodes 232A, 232B used to deliver stimulation pulses. Alternatively, in other cases, the particular combination of electrodes used for electrophysiological sensing includes at least one of the same electrodes as a set of electrodes used to deliver stimulation pulses to patient 105. Sensing circuitry 206 may provide signals to an analog-to-digital converter, for conversion into a digital signal for processing, analysis, storage, or output by processing circuitry 210.

Sensors 222 may be configured to determine motion information. For example, sensors 222 may comprise an accelerometer. Examples of posture information may include accelerometer information indicating an acceleration of medical device 200 along one, two, or three axes, which may represent an acceleration of patient 105. Processing circuitry 210 may determine a current activity level for patient 105 based on the posture information (e.g., accelerometer information). In some examples, posture information may comprise one or more of a velocity, orientation (e.g., with respect to ground), or a position of IMD 200, which may correspond to posture information for patient 105.

Processing circuitry 210 may determine a position state of patient 105. For example, processing circuitry 210 may determine the position state of patient 105 using posture information. For instance, in response to determining that a current acceleration vector generated by an accelerometer of sensors 222 corresponds to (e.g., matches) an acceleration vector associated with a supine position for patient 105, processing circuitry 210 may determine that the position state of patient 105 is supine. Examples of position states of patient 105 may include supine position, prone position, standing, sitting, and/or other position states.

In some examples, processing circuitry 210 may determine an amount of motion of patient 105 based on the posture information (e.g., accelerometer information). For example, processing circuitry 210 may determine that patient 105 is sleeping when the posture information (e.g., accelerometer information) indicates an activity level that is less than a sleep threshold. In some examples, processing circuitry 210 may determine a sleep position during a period of time (e.g., a day) for patient 105 based on the posture information (e.g., accelerometer information). Similarly, processing circuitry 210 may determine that patient 105 is not sleeping (e.g., awake) when the posture information (e.g., accelerometer information) indicates an activity level that is greater than an awake threshold.

Sensors 222 may be configured to determine a heart rate of patient 105. For example, sensor 222 may comprise circuitry configured to detect electrical signals generated by a cardiovascular system of patient 105 to determine the heart rate of patient 105. In some examples, sensor 222 may comprise circuitry configured to detect a motion caused by the cardiovascular system of patient 105 to determine the heart rate of patient 105. In some instances, telemetry circuitry 208 may receive an indication of the heart rate of patient 105 (e.g., that is detected or estimated by another device).

Evoked compound action potentials (ECAPs) are a measure of neural recruitment because each ECAP signal represents the superposition of electrical potentials generated from a population of axons firing in response to an electrical stimulus (e.g., a stimulation pulse). Changes in a characteristic (e.g., an amplitude of a portion of the signal, absolute amplitude between two peaks such as the N1 and P2 peaks, or area under the curve of the signal) of an ECAP signals occur as a function of how many axons have been activated by the delivered stimulation pulse. For a given set of parameter values that define the stimulation pulse and a given distance between the electrodes and target nerve, the detected ECAP signal may have a certain characteristic value (e.g., amplitude). Therefore, a system can determine that the distance between electrodes and nerves has increased or decreased, or that the stimulation is eliciting a different level of nerve activity if the patient has not moved, in response to determining that the measured ECAP characteristic value has increased or decreased. For example, if the set of parameter values stays the same and the ECAP characteristic value of amplitude increases, the system can determine that the distance between electrodes and the nerve has decreased.

Sensors 222 may be configured to generate an evoked compound action potential for patient 105. For example, sensors 222 may generate an ECAP for patient 105. Processing circuitry 210 may generate a range of values using an evoked compound action potential. For example, processing circuitry 210 may identify, using ECAP information, parameter values that elicit discomfort in patient 105 and/or that stimulation circuitry 202 does not provide enough stimulation to patient 105 to provide symptom relief. For instance, processing circuitry 210 may identify, using ECAP information, a range of threshold values where the parameter values do not elicit discomfort in patient 105 and where the parameter values result in stimulation circuitry 202 providing enough stimulation to patient 105 to provide symptom relief. In this way, processing circuitry 210 may ignore adjustments outside of the range of threshold values and/or ensure that the representative value is within the range of threshold values.

Telemetry circuitry 208 may support wireless communication between IMD 200 and an external programmer (not shown in FIG. 2) or another computing device under the control of processing circuitry 210. Processing circuitry 210 of IMD 200 may receive, as updates to programs, values for various stimulation parameters such as amplitude and electrode combination, from the external programmer via telemetry circuitry 208. Telemetry circuitry 208 in IMD 200, as well as telemetry circuits in other devices and systems described herein, such as the external programmer, may accomplish communication by radiofrequency (RF) communication techniques. In addition, telemetry circuitry 208 may communicate with an external medical device programmer (not shown in FIG. 2) via proximal inductive interaction of IMD 200 with the external programmer. The external programmer may be one example of external programmer 150 of FIG. 1. Accordingly, telemetry circuitry 208 may send information to the external programmer on a continuous basis, at periodic intervals, or upon request from IMD 110 or the external programmer.

Processing circuitry 210 may include any one or more of a microprocessor, a controller, a digital signal processor (DSP), an application specific integrated circuit (ASIC), a field-programmable gate array (FPGA), discrete logic circuitry, or any other processing circuitry configured to provide the functions attributed to processing circuitry 210 herein may be embodied as firmware, hardware, software or any combination thereof. As shown, processing circuitry 210 may comprise a representative value unit 245 that may comprise circuitry and/or software instructions. The software instructions associated with representative value unit 245 may be stored, for example, at storage device 212.

Storage device 212 may be configured to store information within IMD 200 during operation. Storage device 212 may include a computer-readable storage medium or computer-readable storage device. In some examples, storage device 212 includes one or more of a short-term memory or a long-term memory. Storage device 212 may include, for example, random access memories (RAM), dynamic random access memories (DRAM), static random access memories (SRAM), magnetic discs, optical discs, flash memories, or forms of electrically programmable memories (EPROM) or electrically erasable and programmable memories (EEPROM). In some examples, storage device 212 is used to store data indicative of instructions for execution by processing circuitry 210, such as, for example, instructions associated with representative value unit 245.

Power source 224 may be configured to deliver operating power to the components of IMD 200. Power source 224 may include a battery and a power generation circuit to produce the operating power. In some examples, the battery is rechargeable to allow extended operation. In some examples, power source 224 may be configured to recharge a battery through proximal inductive interaction between an external charger and an inductive charging coil within IMD 200. Power source 224 may include any one or more of a plurality of different battery types, such as nickel cadmium batteries and lithium ion batteries.

In accordance with the techniques of the disclosure, processing circuitry 210 may receive, using telemetry circuitry 208, an indication of a plurality of user inputs, each user input of the plurality of user inputs indicating a respective value of a plurality of values for a stimulation parameter that at least partially defines therapy provided to patient 105 in a posture state. For example, patient 105 may interact with a touchscreen of external programmer 150 to specify a titration of the amplitude a number of times while the patient is laying down. Processing circuitry 210 may determine, using sensors 222 (e.g., an accelerometer) the posture state of patient 105 based on posture information. In some examples, processing circuitry 210 may store an indication of each respective value indicated by user inputs and an indication of the posture state (e.g., an acceleration vector, a state posture identifier, etc) at storage device 212.

External programmer 150 may output an indication of each interaction to IMD 200. Processing circuitry 210 may control stimulation circuitry 202 to provide therapy with each respective value of the plurality of values. For instance, processing circuitry 210 may control stimulation circuitry 202 to provide therapy with at 2.0 mA, and then, control stimulation circuitry 202 to provide therapy with at 2.2 mA, and so on. In this way, processing circuitry 210 may control stimulation circuitry 202 to provide therapy with at a user requested value.

Representative value unit 245 may determine a representative value for the stimulation parameter based on the plurality of values for the stimulation parameter that at least partially defines therapy provided to patient 105 in the posture state. For example, representative value unit 245 may average (e.g., determine a mean or mode, weighted average, or other modulated average) the titrations of the amplitude 10 times to an amplitude of 2.3 mA. In some examples, representative value unit 245 may be omitted. For instance, one or more steps performed by representative value unit 245 may be performed by representative value unit 345 of external programmer 300 or another device.

Processing circuitry 210 may control, based on a determination that patient 105 is in the posture state once again (e.g., after leaving the posture state and entering a different posture state), IMD 110 to provide the therapy using the representative value for the stimulation parameter. For example, processing circuitry 210 may control, based on a determination that patient 105 is laying down, stimulation circuitry 202 to provide therapy with an amplitude of 2.3 mA.

In some examples, processing circuitry 210 may omit one or more “outlier” values. For example, processing circuitry 210 may receive a second user input indicating a second value for the stimulation parameter that at least partially defines therapy provided to patient 105 in the posture state. In this example, processing circuitry 210 may determine that an indication of a patient reported outcome (e.g., received by external programmer 150) indicates that the second value is an outlier value. For instance, external programmer 150 may output the indication that the second value is an outlier value in response to receiving an indication of agreement from a user that is responsive to a prompt “Did you over exercise?”. In some examples, external programmer 150 may determine that the second value is an outlier value when the second value is a patient adjustment that is a percentage or standard deviation from an average value. Processing circuitry 210 may determine to omit the second value from being used to determine the representative value based on the determination that a patient reported outcome indicated that the second value is the outlier value.

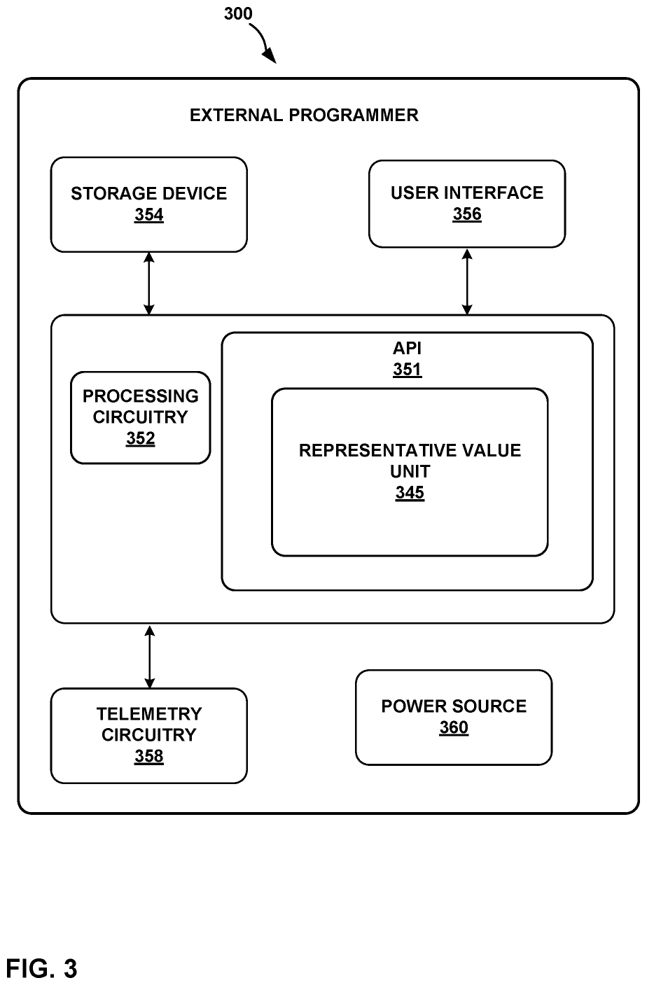

FIG. 3 is a block diagram illustrating an example configuration of components of an example external programmer 300. External programmer 300 may be an example of external programmer 150 (e.g., an external patient programmer or an external clinician programmer) of FIG. 1. Although external programmer 300 may generally be described as a hand-held device, external programmer 300 may be a larger portable device or a more stationary device. In addition, in other examples, external programmer 300 may be included as part of an external charging device or include the functionality of an external charging device. As illustrated in FIG. 3, external programmer 300 may include processing circuitry 352, storage device 354, user interface 356, telemetry circuitry 358, and power source 360. Storage device 354 may store instructions that, when executed by processing circuitry 352, cause processing circuitry 352 and external programmer 300 to provide the functionality ascribed to external programmer 300 throughout this disclosure. Each of these components, circuitry, or modules, may include electrical circuitry that is configured to perform some, or all of the functionality described herein. For example, processing circuitry 352 may include one or more processors, such as, one or more microprocessors, DSPs, ASICs, FPGAs, or any other equivalent integrated or discrete logic circuitry, as well as any combinations of such components, configured to perform the processes discussed with respect to processing circuitry 352. External programmer 300 may represent a patient programmer, clinician programmer, or another device.

In general, external programmer 300 includes any suitable arrangement of hardware, alone or in combination with software and/or firmware, to perform the techniques attributed to external programmer 300, and processing circuitry 352, user interface 356, and telemetry circuitry 358 of external programmer 300. In various examples, external programmer 300 may include one or more processors, such as one or more microprocessors, DSPs, ASICs, FPGAs, or any other equivalent integrated or discrete logic circuitry, as well as any combinations of such components. External programmer 300 also, in various examples, may include a storage device 354, such as RAM, ROM, PROM, EPROM, EEPROM, flash memory, a hard disk, a CD-ROM, including executable instructions for causing the one or more processors to perform the actions attributed to them. Moreover, although processing circuitry 352 and telemetry circuitry 358 are described as separate modules, in some examples, processing circuitry 352 and telemetry circuitry 358 are functionally integrated. In some examples, processing circuitry 352 and telemetry circuitry 358 correspond to individual hardware units, such as ASICs, DSPs, FPGAs, or other hardware units.

Storage device 354 (e.g., a storage device) may store instructions that, when executed by processing circuitry 352, cause processing circuitry 352 and external programmer 300 to provide the functionality ascribed to external programmer 300 throughout this disclosure. For example, storage device 354 may include instructions that cause processing circuitry 352 to obtain a parameter set from memory or receive user input and send a corresponding command to IMD 110, or instructions for any other functionality. In addition, storage device 354 may include a plurality of programs, where each program includes a parameter set that defines therapy stimulation or control stimulation. Storage device 354 may also store data received from a medical device (e.g., IMD 110). For example, storage device 354 may store data recorded at a sensing module of the medical device, and storage device 354 may also store data from one or more sensors of the medical device.

Processing circuitry 352 may be configured to control IMD 110 with a program to provide stimulation. For example, processing circuitry 352 may automatically or semi-automatically set or adjust programs at IMD 110 by transmitting, with telemetry circuitry 358, instructions to IMD 110. For instance, in response to a change (e.g., a change indicated by user input, a change sensed by IMD 110, etc.) in activity of a patient (e.g., standing, walking, voiding, etc.), processing circuitry 352 may automatically or semi-automatically set or adjust programs at IMD 110. For instance, processing circuitry 352 may, in response to determining that the patient would not like to void, output instructions to IMD 110 to use a first group stored at IMD 110 for controlled voiding. In this instance, processing circuitry 352 may, in response to determining that the patient would like to void, output instructions to IMD 110 to use a new group or program stored at IMD 110 for controlled voiding.

User interface 356 may include a button or keypad, lights, a speaker for voice commands, a display, such as a liquid crystal (LCD), light-emitting diode (LED), or organic light-emitting diode (OLED). In some examples the display includes a touch screen. User interface 356 may be configured to display any information related to the delivery of electrical stimulation. User interface 356 may also receive user input (e.g., indication of when the patient perceives a stimulation pulse) via user interface 356. The input may be, for example, in the form of pressing a button on a keypad or selecting an icon from a touch screen. The input may request starting or stopping electrical stimulation, the input may request a new spatial electrode pattern or a change to an existing spatial electrode pattern, of the input may request some other change to the delivery of electrical stimulation.

Telemetry circuitry 358 may support wireless communication between the medical device and external programmer 300 under the control of processing circuitry 352. Telemetry circuitry 358 may also be configured to communicate with another computing device via wireless communication techniques, or direct communication through a wired connection. In some examples, telemetry circuitry 358 provides wireless communication via an RF or proximal inductive medium. In some examples, telemetry circuitry 358 includes an antenna, which may take on a variety of forms, such as an internal or external antenna.

Examples of local wireless communication techniques that may be employed to facilitate communication between external programmer 300 and IMD 110 include RF communication according to the 802.11 or Bluetooth® specification sets or other standard or proprietary telemetry protocols. In this manner, other external devices may be capable of communicating with external programmer 300 without needing to establish a secure wireless connection. As described herein, telemetry circuitry 358 may be configured to transmit a spatial electrode movement pattern or other stimulation parameter values to IMD 110 for delivery of electrical stimulation therapy.

Power source 360 is configured to deliver operating power to the components of external programmer 300. Power source 360 may include a battery and a power generation circuit to produce the operating power. In some examples, the battery is rechargeable to allow extended operation. Recharging may be accomplished by electrically coupling power source 360 to a cradle or plug that is connected to an alternating current (AC) outlet. In addition, recharging may be accomplished through proximal inductive interaction between an external charger and an inductive charging coil within external programmer 300. In other examples, traditional batteries (e.g., nickel cadmium or lithium ion batteries) may be used. In addition, external programmer 300 may be directly coupled to an alternating current outlet to operate.

Processing circuitry 352 may implement API 351 to facilitate the control of IMD 110. API 351 may include representative value unit 345. Representative value unit 345 may be configured to determine a representative value for the stimulation parameter based on the plurality of values for the stimulation parameter that at least partially defines therapy provided to patient 105 in the posture state. For example, representative value unit 345 may average (e.g., determine a mean or mode) of the titrations of the amplitude 10 times to an amplitude of 2.3. In some examples, representative value unit 345 may be omitted. For instance, one or more steps performed by representative value unit 345 may be performed by representative value unit 245 of IMD 200 or another device.

In accordance with the techniques of the disclosure, processing circuitry 352, using user interface 356, may receive an indication of a plurality of user inputs, each user input of the plurality of user inputs indicating a respective value of a plurality of values for a stimulation parameter that at least partially defines therapy provided to patient 105 in a posture state. For example, patient 105 may interact with a touchscreen of user interface 356 to specify a titration of the amplitude a number of times while patient 105 is laying down. Processing circuitry 352 may determine, using sensors (e.g., an accelerometer) of IMD 110, the posture state of patient 105 based on posture information. In some examples, processing circuitry 352 may store an indication of each respective value indicated by user inputs and an indication of the posture state (e.g., an acceleration vector, a state posture identifier, etc) at storage device 354.

Processing circuitry 352 may control IMD 110 to provide therapy with each respective value of the plurality of values. For instance, processing circuitry 352 may control, using telemetry circuitry 358, IMD 110 to provide therapy with at 2.0, and then, control IMD 110 to provide therapy with at 2.2, and so on. In this way, processing circuitry 352 may control IMD 110 to provide therapy with at a user requested value.

Representative value unit 345 may determine a representative value for the stimulation parameter based on the plurality of values for the stimulation parameter that at least partially defines therapy provided to patient 105 in the posture state. For example, representative value unit 345 may average (e.g., determine a mean or mode) of the titrations of the amplitude 10 times to an amplitude of 2.3 mA. Processing circuitry 352 may control, based on a determination that patient 105 is in the posture state, IMD 110 to provide the therapy using the representative value for the stimulation parameter. For example, processing circuitry 352 may control, based on a determination that patient 105 is in a posture (e.g., laying down), IMD 110 to provide therapy with an amplitude of 2.3 mA.

In some examples, processing circuitry 352 may omit one or more “outlier” values. For example, processing circuitry 352, using user interface 356, may receive a second user input indicating a second value for the stimulation parameter that at least partially defines therapy provided to patient 105 in the posture state. In this example, processing circuitry 352 may determine that the second value is a potential outlier value based on the plurality of first values and the second value. For instance, processing circuitry 352 may determine that the second value (e.g., 4.0 mA) is a potential outlier value based on a determination that the second value is outside of a range of values (e.g., 1.8 mA-2.7 mA) determined using the plurality of first values. In this example, processing circuitry 352 may output, using user interface 356, an indication prompting whether the second value the outlier value based on the determination that second value is a potential outlier value. In this example, processing circuitry 352 may receive, with user interface 356, an indication of a patient reported outcome that indicates that the second value is an outlier value after outputting the indication prompting whether the second value is associated with the outlier value (e.g., not normal for the posture). For instance, the patient reported outcome may indicate that patient 105 has over exercised. In some instances, the patient reported outcome may indicate that patient 105 is sick. In this example, processing circuitry 352 may determine to omit the second value from being used to determine the representative value based on the determination that a patient reported outcome indicated that the second value is the outlier value.

The architecture of external programmer 300 illustrated in FIG. 3 is shown as an example. The techniques as set forth in this disclosure may be implemented in the example external programmer 300 of FIG. 3, as well as other types of systems not described specifically herein. Nothing in this disclosure should be construed so as to limit the techniques of this disclosure to the example architecture illustrated by FIG. 3.

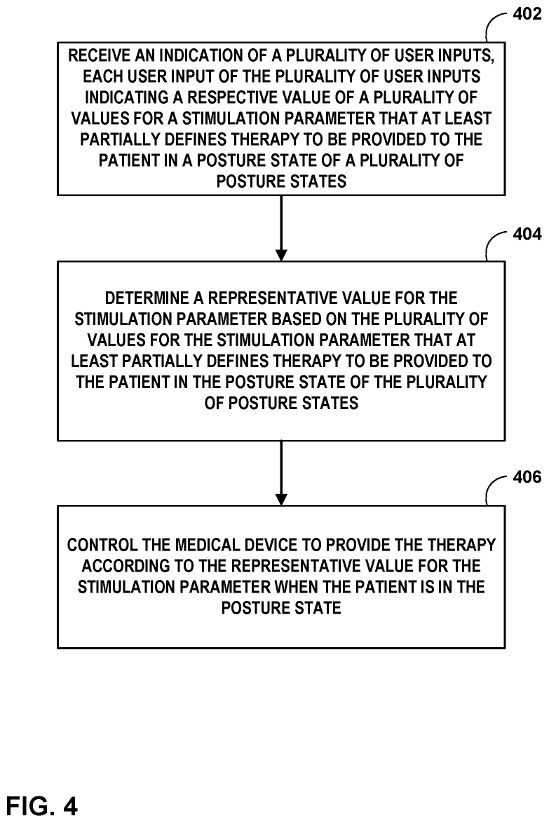

FIG. 4 is a flow diagram illustrating a process for determining a representative value for a stimulation parameter that at least partially defines a therapy provided to a patient, in accordance with one or more techniques of this disclosure. FIG. 4 is discussed with reference to FIGS. 1-3 for example purposes only. In the following example, external programmer 150 performs 402-406 of FIG. 4. However, in other examples, other devices (e.g., IMD 110) or some combination of devices may perform the process of FIG. 4 as explained in further detail below. In the following examples, IMD 110 is used as a medical device. However, in some examples, an external medical device may be used instead of IMD 110. In some examples, external device 150 may comprise a mobile device.

Processing circuitry 352 may receive, using user interface 356, an indication of a plurality of user inputs, each user input of the plurality of user inputs indicating a respective value of a plurality of values for a stimulation parameter that at least partially defines therapy provided to patient 105 in a posture state of a plurality of posture states (402). For example, processing circuitry 352 may receive the plurality of user inputs as patient adjustments over time for a posture state. For instance, patient 105 may interact with a touchscreen of user interface 356 to specify a titration of the amplitude while patient 105 is laying down. Processing circuitry 352 may determine, using sensors (e.g., an accelerometer) of IMD 110, the posture state of patient 105 based on posture information. In some examples, processing circuitry 352 may store an indication of each respective value indicated by user inputs and an indication of the posture state (e.g., an acceleration vector, a state posture identifier, etc) at storage device 354.

Before processing circuitry 352 controls IMD 110 to provide the therapy using a representative value, processing circuitry 352 may control IMD 110 to provide the therapy using the respective value indicated by a user input of the plurality of user inputs. For example, processing circuitry 352 may control, using telemetry circuitry 358, IMD 110 to provide therapy with at 2.0, and then, control IMD 110 to provide therapy with at 2.2 mA, and so on. In this way, processing circuitry 352 may control IMD 110 to provide therapy with at a user requested value. In some examples, each user input and respective value may be associated with a respective time range. For example, processing circuitry 352 may receive, using user interface 356, a first user input of the plurality of user inputs during a first time and receive a second user input of the plurality of user inputs during a second time that is different (e.g., different overlapping ranges or non-overlapping ranges) from the first time range.

Processing circuitry 352 may determine the plurality of values for the stimulation parameter based on the posture state of patient 105. For example, processing circuitry 352 may determine, for a user input, posture information corresponding to a time range of when the user input was received. In this example, processing circuitry 352 may determine that the posture information corresponds to the posture state. Processing circuitry 352 may determine the plurality of user inputs to include the user input based on the determination that the posture information corresponds to the posture state. For instance, processing circuitry 352 may determine a plurality of first user inputs corresponding to a first posture state (e.g., sitting) and may determine a plurality of second user inputs corresponding to a second posture state (e.g., laying down). Sensors 222 may generate posture information. For instance, an accelerometer of sensors 22 may generate acceleration information indicated by the posture information.

Representative value unit 345 may determine a representative value for the stimulation parameter based on the plurality of values for the stimulation parameter that at least partially defines therapy provided to patient 105 in the posture state of the plurality of posture states (404). For example, representative value unit 345 may average (e.g., determine a mean or mode) the titrations of the amplitude to an amplitude of 2.3 mA. In some examples, after an initial training using a first set of N number of titrations of the amplitude, representative value unit 345 may continue to update the representative value (e.g., a rolling value), for example, using a most recent set of M number of titrations (where M is a positive integer) of the amplitude while the patient is in the posture state of the plurality of different posture states (e.g., sitting, standing, walking, or laying down in different positions such as supine and prone posture states).

Processing circuitry 352 may control IMD 110 to provide the therapy according to the representative value for the stimulation parameter when patient 105 is in the posture state (406). For example, processing circuitry 352 may control IMD 110 to provide therapy with an amplitude of 2.3 mA when patient 105 is laying down. In some examples, patient 105 may adjust the amplitude as needed, which processing circuitry 352 may further use to provide therapy and/or to update the representative value.

In some examples, processing circuitry 352 may omit one or more “outlier” values. For example, processing circuitry 352 may receive, using user interface 356, a second user input indicating a second value for the stimulation parameter that at least partially defines therapy provided to patient 105 in the posture state. In this example, processing circuitry 352 may determine that the second value is a potential outlier value based on the plurality of first values and the second value. For instance, processing circuitry 352 may determine that the second value (e.g., 4.0 mA) is a potential outlier value based on a determination that the second value is outside of a range of values (e.g., 1.8 mA-2.7 mA) determined using the plurality of first values. In this example, processing circuitry 352 may output, using user interface 356, an indication prompting whether the second value the outlier value based on the determination that second value is a potential outlier value. In this example, processing circuitry 352 may receive, with user interface 356, an indication of a patient reported outcome (PRO) that indicates that the second value is an outlier value after outputting the indication prompting whether the second value is associated with the outlier value. For instance, the patient reported outcome may indicate that patient 105 has over exercised. In this example, processing circuitry 352 may determine to omit the second value from being used to determine the representative value based on the determination that a patient reported outcome indicated that the second value is the outlier value.

Processing circuitry 352 may determine the representative value to be within a range of values determined using an evoked compound action potential for patient 105. For example, processing circuitry 352 may receive, using telemetry circuitry 358, an indication of an evoked compound action potential for patient 105. In this example, processing circuitry 352 may determine a range of values using an evoked compound action potential. Processing circuitry 352 may determine the representative value to be within a range of values determined using an evoked compound action potential. For instance, processing circuitry 352 may reduce the representative value to be a maximum of the range of values determined using an evoked compound action potential when an average value of the plurality of values for a stimulation parameter is greater than the range of values. Similarly, processing circuitry 352 may increase the representative value to be a minimum of the range of values determined using an evoked compound action potential when an average value of the plurality of values for a stimulation parameter is less than the range of values. In this way, processing circuitry 210 may ignore adjustments outside of the range of threshold values and/or ensure that the representative value is within the range of threshold values.

In some examples, processing circuitry 352 may be configured to “micro-titrate” based on a heart rate of patient 105 and/or a motion for patient 105. For example, processing circuitry 352 may determine that patient 105 is sleeping when the heart rate of patient 105 is less than a sleeping threshold. In response to a determination that the heart rate of patient 105 is less than a sleeping threshold, processing circuitry 352 may determine the representative value to be less than an average value determined based on the plurality of values based on a determination that the heart rate of patient 105 is less than a threshold heart rate value. For instance, processing circuitry 352 may determine, based on a determination that the heart rate of patient 105 is less than a sleeping threshold, that patient 105 is associated with a sleeping state. In this example, processing circuitry 352 may determine the representative value to be less than the average value (e.g., less than 99%, less than 95%, less than 90% of the average value) in response to the determination that the patient is associated with the sleeping state. For instance, processing circuitry 352 may determine the representative value to be 95% of an average value of the plurality of values in response to the determination that the patient is associated with the sleeping state. Similarly, processing circuitry 352 may determine that patient 105 is not sleeping (e.g., awake) when the heart rate of patient 105 is greater than an awake threshold. In this example, processing circuitry 352 may determine, based on a determination that the heart rate of patient 105 is greater than an awake threshold, the representative value to be the average value determined based on the plurality of values (e.g., a mean of the plurality of values).

Processing circuitry 352 may determine the representative value based on a motion (e.g., a degree of movement) for patient 105 indicated by posture information. For example, processing circuitry 352 may determine that patient 105 is associated with a sleeping state when the motion for patient 105 is less than a sleeping threshold. In response to a determination that the patient 105 is associated with the sleeping state, processing circuitry 352 may determine the representative value to be less than (e.g., less than 99%, less than 95%, less than 90% of the average value) an average value determined based on the plurality of values. For instance, processing circuitry 352 may determine, based on a determination that the patient 105 is associated with the sleeping state, the representative value to be 95% of an average value of the plurality of values. Similarly, processing circuitry 352 may determine that patient 105 is not sleeping (e.g., awake) when the motion information for patient 105 is greater than an awake threshold. In this example, processing circuitry 352 may determine, based on a determination that the motion information for patient 105 is greater than an awake threshold, the representative value to be the average value determined based on the plurality of values (e.g., a mean of the plurality of values).

The following examples are a non-limiting list of clauses in accordance with one or more techniques of this disclosure.

Clause 1. A system comprising: telemetry circuitry configured for communication between a medical device and an external device associated with the medical device; and processing circuitry configured to: receive an indication of a plurality of user inputs, each user input of the plurality of user inputs indicating a respective value of a plurality of values for a stimulation parameter that at least partially defines therapy provided to the patient in a posture state of a plurality of posture states; determine a representative value for the stimulation parameter based on the plurality of values for the stimulation parameter that at least partially defines therapy provided to the patient in the posture state; and control the medical device to provide the therapy according to the representative value for the stimulation parameter when the patient is in the posture state.

Clause 2. The system of clause 1, wherein the processing circuitry is configured to, for each respective user input of the plurality of user inputs: determine posture information for the patient corresponding to a time that the respective user input was received; determine, based on the posture information, the posture state; and determine, based on the determination that the posture information corresponds to the posture state, that the respective user input is to be associated with the plurality of user inputs for the posture state.

Clause 3. The system of clause 2, further comprising an accelerometer configured to generate the posture information.

Clause 4. The system of any of clauses 1-3, wherein, to receive the indication of the plurality of user inputs, the processing circuitry is configured to: receive a first user input of the plurality of user inputs during a first time; and receive a second user input of the plurality of user inputs during a second time that is different from the first time.

Clause 5. The system of any of clauses 1-4, wherein, to determine the representative value for the stimulation parameter, the processing circuitry is configured to average the plurality of values for the stimulation parameter.

Clause 6. The system of any of clauses 1-5, wherein the plurality of user inputs comprises a plurality of first user inputs and the plurality of values comprises a plurality of first values, and wherein the processing circuitry is further configured to: receive a second user input indicating a second value for the stimulation parameter that at least partially defines therapy provided to the patient in the posture state; determine that an indication of a patient reported outcome indicates that the second value is an outlier value; and determine to omit the second value from the plurality of values based on the determination that a patient reported outcome indicated that the second value is the outlier value.

Clause 7. The system of clause 6, wherein the processing circuitry is configured to: determine that the second value is a potential outlier value based on the plurality of first values and the second value; output an indication prompting whether the second value is associated with an outlier value based on the determination that second value is a potential outlier value; and subsequent to outputting the indication prompting whether the second value is associated with the outlier value, receive the indication of the patient reported outcome.

Clause 8. The system of any of clauses 1-7, wherein, to determine the representative value for the stimulation parameter, the processing circuitry is configured to determine the representative value to be within a range of values determined based on an evoked compound action potential sensed for the posture state.

Clause 9. The system of any of clauses 1-8, wherein, to determine the representative value for the stimulation parameter, the processing circuit is configured to determine the representative value based on a heart rate of the patient.

Clause 10. The system of clause 9, wherein, to determine the representative value for the stimulation parameter, the processing circuit is configured to: determine an average value based on the based on the plurality of values for the stimulation parameter; determine that the patient is associated with a sleeping state based on that the heart rate of the patient being less than a threshold heart rate value; and determine the representative value to be less than the average value in response to the determination that the patient is associated with the sleeping state.

Clause 11. The system of claim any of clauses 1-8, wherein, to determine the representative value for the stimulation parameter, the processing circuit is configured to: determine an average value based on the based on the plurality of values for the stimulation parameter; and determine the representative value to be less than the average value based on a determination that the posture information of the patient indicates a motion that is less than a threshold motion value.

Clause 12. The system of any of clauses 1-11, wherein the processing circuit is configured to, before the processing circuitry controls the medical device to provide the therapy using the representative value, control the medical device to provide the therapy using the respective value indicated by a user input of the plurality of user inputs.

Clause 13. The system of any of clauses 1-12, wherein the external device comprises a mobile device.

Clause 14. The system of any of clauses 1-13, wherein the medical device comprises the processing circuitry.

Clause 15. The system of any of clauses 1-13, wherein the external device comprises the processing circuitry.

Clause 16. The system of any of clauses 1-13, wherein the external device and the medical device comprise the processing circuitry.

Clause 17. The system of any of clauses 1-16, wherein the medical device comprises an implantable medical device.

Clause 18. The system of any of clauses 1-17, further comprising the medical device.

Clause 19. The system of clauses 1-18, wherein the medical device is configured to provide the therapy as one or more of deep brain stimulation (DBS), spinal cord stimulation (SCS), sacral neuromodulation (SNS), targeted drug delivery (TDD), pelvic stimulation, gastric stimulation, or peripheral nerve field stimulation (PNFS).

Clause 20. A method comprising: receiving, by one or more processors, an indication of a plurality of user inputs, each user input of the plurality of user inputs indicating a respective value of a plurality of values for a stimulation parameter that at least partially defines therapy provided to the patient in a posture state of a plurality of posture states; determining, by the one or more processors, a representative value for the stimulation parameter based on the plurality of values for the stimulation parameter that at least partially defines therapy provided to the patient in the posture state; and controlling, by the one or more processors, the medical device to provide the therapy according to the representative value for the stimulation parameter when the patient is in the posture state.

Clause 21. The method of clause 20, further comprising, for each respective user input of the plurality of user inputs: determining, by the one or more processors, posture information for the patient corresponding to a time that the respective user input was received; determining, by the one or more processors, based on the posture information, the posture state; and determining, by the one or more processors, based on the determination that the posture information corresponds to the posture state, that the respective user input is to be associated with the plurality of user inputs for the posture state.

Clause 22. The method of clause 21, wherein an accelerometer is configured to generate the posture information.

Clause 23. The method of any of clauses 20-22, wherein receiving the indication of the plurality of user inputs comprises receiving a first user input of the plurality of user inputs during a first time; and receiving a second user input of the plurality of user inputs during a second time that is different from the first time.

Clause 24. The method of any of clauses 20-23, wherein determining the representative value for the stimulation parameter comprises averaging the plurality of values for the stimulation parameter.

Clause 25. The method of any of clauses 20-24, wherein the plurality of user inputs comprises a plurality of first user inputs and the plurality of values comprises a plurality of first values, the method further comprising: receiving, by the one or more processors, a second user input indicating a second value for the stimulation parameter that at least partially defines therapy provided to the patient in the posture state; determining, by the one or more processors, that an indication of a patient reported outcome indicates that the second value is an outlier value; and determining, by the one or more processors, to omit the second value from the plurality of values based on the determination that a patient reported outcome indicated that the second value is the outlier value.

Clause 26. The method of clause 25, further comprising: determining, by the one or more processors, that the second value is a potential outlier value based on the plurality of first values and the second value; outputting, by the one or more processors, an indication prompting whether the second value is associated with an outlier value based on the determination that second value is a potential outlier value; and subsequent to outputting the indication prompting whether the second value is associated with the outlier value, receiving, by the one or more processors, the indication of the patient reported outcome.

Clause 27. The method of any of clauses 20-26, wherein determining the representative value for the stimulation parameter comprises determining the representative value to be within a range of values determined based on an evoked compound action potential sensed for the posture state.

Clause 28. The method of any of clauses 20-27, wherein determining the representative value for the stimulation parameter comprises determining the representative value based on a heart rate of the patient.

Clause 29. The method of clause 28, wherein determining the representative value for the stimulation parameter comprises determining an average value based on the based on the plurality of values for the stimulation parameter; determining that the patient is associated with a sleeping state based on that the heart rate of the patient being less than a threshold heart rate value; and determining the representative value to be less than the average value in response to the determination that the patient is associated with the sleeping state.

Clause 30. The method of claim any of clauses 20-27, wherein determining the representative value for the stimulation parameter comprises: determining an average value based on the based on the plurality of values for the stimulation parameter; and determining the representative value to be less than the average value based on a determination that the posture information of the patient indicates a motion that is less than a threshold motion value.

Clause 31. The method of any of clauses 20-30, further comprising, before the controlling of the medical device to provide the therapy using the representative value, controlling the medical device to provide the therapy using the respective value indicated by a user input of the plurality of user inputs.

Clause 32. The method of any of clauses 20-31, wherein the external device comprises a mobile device.

Clause 33. The method of any of clauses 20-32, wherein the medical device comprises the one or more processors.

Clause 34. The method of any of clauses 20-32, wherein the external device comprises the one or more processors.

Clause 35. The method of any of clauses 20-32, wherein the external device and the medical device comprise the one or more processors.

Clause 36. The method of any of clauses 20-35, wherein the medical device comprises an implantable medical device.

Clause 37. The method of clauses 20-36, wherein the medical device is configured to provide the therapy as one or more of deep brain stimulation (DBS), spinal cord stimulation (SCS), sacral neuromodulation (SNS), targeted drug delivery (TDD), pelvic stimulation, gastric stimulation, or peripheral nerve field stimulation (PNFS).

Clause 38. A computer-readable storage medium having stored thereon instructions that, when executed, cause processing circuitry to: receive an indication of a plurality of user inputs, each user input of the plurality of user inputs indicating a respective value of a plurality of values for a stimulation parameter that at least partially defines therapy provided to the patient in a posture state of a plurality of posture states; determine a representative value for the stimulation parameter based on the plurality of values for the stimulation parameter that at least partially defines therapy provided to the patient in the posture state; and control the medical device to provide the therapy according to the representative value for the stimulation parameter when the patient is in the posture state.

While the above examples discussed determining the representative value based on only heart rate and based on only the motion for patient 105, in some examples, processing circuitry 352 may determine the representative value based on a combination of both heart rate and the motion for patient 105. For example, processing circuitry 352 may determine the representative value to be less than an average value in response to a determination that both the heart rate of patient 105 is less than a sleeping threshold for heart rate and the posture information for patient 105 is less than a sleeping threshold for motion.

While examples described herein may be directed to spinal cord stimulation, techniques described herein may be applied to any stimulation device may deliver electrical stimulation therapy via electrodes, e.g., carried by one or more leads, positioned proximate to target locations associated with the brain, the spinal cord, pelvic nerves, tibial nerves, peripheral nerves, the gastrointestinal tract, or elsewhere within a patient. Stimulation proximate the spinal cord, proximate the sacral nerve, within the brain, and proximate peripheral nerves is often referred to as spinal cord stimulation (SCS), sacral neuromodulation (SNM), deep brain stimulation (DBS), and peripheral nerve stimulation (PNS), respectively.

It is to be recognized that depending on the example, certain acts or events of any of the techniques described herein can be performed in a different sequence, may be added, merged, or left out altogether (e.g., not all described acts or events are necessary for the practice of the techniques). Moreover, in certain examples, acts or events may be performed concurrently, e.g., through multi-threaded processing, interrupt processing, or multiple processors, rather than sequentially.

In one or more examples, the functions described may be implemented in hardware, software, firmware, or any combination thereof. If implemented in software, the functions may be stored on or transmitted over as one or more instructions or code on a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable medium.

By way of example, and not limitation, such computer-readable storage media can comprise RAM, ROM, EEPROM, CD-ROM or other optical disk storage, magnetic disk storage, or other magnetic storage devices, flash memory, or any other medium that can be used to store desired program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transitory media, but are instead directed to non-transitory, tangible storage media. Disk and disc, as used herein, includes compact disc (CD), laser disc, optical disc, digital versatile disc (DVD), floppy disk and Blu-ray disc, where disks usually reproduce data magnetically, while discs reproduce data optically with lasers. Combinations of the above should also be included within the scope of computer-readable media.

Instructions may be executed by one or more processors, such as one or more digital signal processors (DSPs), general purpose microprocessors, application specific integrated circuits (ASICs), field programmable gate arrays (FPGAs), or other equivalent integrated or discrete logic circuitry. Accordingly, the terms “processor” and “processing circuitry,” as used herein may refer to any of the foregoing structures or any other structure suitable for implementation of the techniques described herein. In addition, in some aspects, the functionality described herein may be provided within dedicated hardware and/or software modules configured for encoding and decoding, or incorporated in a combined codec. Also, the techniques could be fully implemented in one or more circuits or logic elements.

The techniques of this disclosure may be implemented in a wide variety of devices or apparatuses, including a wireless handset, an integrated circuit (IC) or a set of ICs (e.g., a chip set). Various components, modules, or units are described in this disclosure to emphasize functional aspects of devices configured to perform the disclosed techniques, but do not necessarily require realization by different hardware units. Rather, as described above, various units may be combined in a codec hardware unit or provided by a collection of interoperative hardware units, including one or more processors as described above, in conjunction with suitable software and/or firmware.

Various examples have been described. These and other examples are within the scope of the following claims.

What is claimed is:

1. A system comprising:
telemetry circuitry configured for communication between a medical device and an external device associated with the medical device; and
processing circuitry configured to:
receive an indication of a plurality of user inputs, each user input of the plurality of user inputs indicating a respective value of a plurality of values for a stimulation parameter that at least partially defines therapy provided to the patient in a posture state of a plurality of posture states;
determine a representative value for the stimulation parameter based on the plurality of values for the stimulation parameter that at least partially defines therapy provided to the patient in the posture state; and
control the medical device to provide the therapy according to the representative value for the stimulation parameter when the patient is in the posture state.

2. The system of claim 1, wherein the processing circuitry is configured to, for each respective user input of the plurality of user inputs:
determine posture information for the patient corresponding to a time that the respective user input was received;
determine, based on the posture information, the posture state; and
determine, based on the determination that the posture information corresponds to the posture state, that the respective user input is to be associated with the plurality of user inputs for the posture state.

3. The system of claim 2, further comprising an accelerometer configured to generate the posture information.

4. The system of claim 1, wherein, to receive the indication of the plurality of user inputs, the processing circuitry is configured to:
receive a first user input of the plurality of user inputs during a first time; and
receive a second user input of the plurality of user inputs during a second time that is different from the first time.

5. The system of claim 1, wherein, to determine the representative value for the stimulation parameter, the processing circuitry is configured to average the plurality of values for the stimulation parameter.

6. The system of claim 1, wherein the plurality of user inputs comprises a plurality of first user inputs and the plurality of values comprises a plurality of first values, and wherein the processing circuitry is further configured to:
receive a second user input indicating a second value for the stimulation parameter that at least partially defines therapy provided to the patient in the posture state;
determine that an indication of a patient reported outcome indicates that the second value is an outlier value; and
determine to omit the second value from the plurality of values based on the determination that a patient reported outcome indicated that the second value is the outlier value.

7. The system of claim 6, wherein the processing circuitry is configured to:
determine that the second value is a potential outlier value based on the plurality of first values and the second value;
output an indication prompting whether the second value is associated with an outlier value based on the determination that second value is a potential outlier value; and
subsequent to outputting the indication prompting whether the second value is associated with the outlier value, receive the indication of the patient reported outcome.

8. The system of claim 1, wherein, to determine the representative value for the stimulation parameter, the processing circuitry is configured to determine the representative value to be within a range of values determined based on an evoked compound action potential sensed for the posture state.

9. The system of claim 1, wherein, to determine the representative value for the stimulation parameter, the processing circuit is configured to determine the representative value based on a heart rate of the patient.

10. The system of claim 9, wherein, to determine the representative value for the stimulation parameter, the processing circuit is configured to:
determine an average value based on the based on the plurality of values for the stimulation parameter;
determine that the patient is associated with a sleeping state based on that the heart rate of the patient being less than a threshold heart rate value; and
determine the representative value to be less than the average value in response to the determination that the patient is associated with the sleeping state.

11. The system of claim 1, wherein, to determine the representative value for the stimulation parameter, the processing circuit is configured to:
determine an average value based on the based on the plurality of values for the stimulation parameter; and
determine the representative value to be less than the average value based on a determination that the posture information of the patient indicates a motion that is less than a threshold motion value.

12. The system of claim 1, wherein the processing circuit is configured to, before the processing circuitry controls the medical device to provide the therapy using the representative value, control the medical device to provide the therapy using the respective value indicated by a user input of the plurality of user inputs.

13. The system of claim 1, wherein the medical device comprises the processing circuitry.

14. A method comprising:
receiving, by one or more processors, an indication of a plurality of user inputs, each user input of the plurality of user inputs indicating a respective value of a plurality of values for a stimulation parameter that at least partially defines therapy provided to the patient in a posture state of a plurality of posture states;
determining, by the one or more processors, a representative value for the stimulation parameter based on the plurality of values for the stimulation parameter that at least partially defines therapy provided to the patient in the posture state; and
controlling, by the one or more processors, the medical device to provide the therapy according to the representative value for the stimulation parameter when the patient is in the posture state.

15. The method of claim 14, further comprising, for each respective user input of the plurality of user inputs:
determining, by the one or more processors, posture information for the patient corresponding to a time that the respective user input was received;
determining, by the one or more processors, based on the posture information, the posture state; and
determining, by the one or more processors, based on the determination that the posture information corresponds to the posture state, that the respective user input is to be associated with the plurality of user inputs for the posture state.

16. The method of claim 14, wherein receiving the indication of the plurality of user inputs, comprises:
receiving a first user input of the plurality of user inputs during a first time; and
receiving a second user input of the plurality of user inputs during a second time that is different from the first time.

17. The method of claim 14, wherein determining the representative value for the stimulation parameter comprises averaging the plurality of values for the stimulation parameter.

18. The method of claim 14, wherein the plurality of user inputs comprises a plurality of first user inputs and the plurality of values comprises a plurality of first values, the method further comprising:
receiving, by the one or more processors, a second user input indicating a second value for the stimulation parameter that at least partially defines therapy provided to the patient in the posture state;
determining, by the one or more processors, that an indication of a patient reported outcome indicates that the second value is an outlier value; and
determining, by the one or more processors, to omit the second value from the plurality of values based on the determination that a patient reported outcome indicated that the second value is the outlier value.

19. The method of claim 14, wherein determining the representative value for the stimulation parameter comprises determining the representative value to be within a range of values determined based on an evoked compound action potential sensed for the posture state.

20. A computer-readable storage medium having stored thereon instructions that, when executed, cause processing circuitry to:
receive an indication of a plurality of user inputs, each user input of the plurality of user inputs indicating a respective value of a plurality of values for a stimulation parameter that at least partially defines therapy provided to the patient in a posture state of a plurality of posture states;
determine a representative value for the stimulation parameter based on the plurality of values for the stimulation parameter that at least partially defines therapy provided to the patient in the posture state; and
control the medical device to provide the therapy according to the representative value for the stimulation parameter when the patient is in the posture state.
